(19) United States
(12) Patent Application Publication
(10) Pub. No.: US 2007/0082920 A1
(43) Pub. Date: Apr. 12, 2007

(54) NAD+-dependent DNA ligase inhibitors

(76) Inventors: Yongsheng Song, Guilford, CT (US); Yongchun Hou, Mentor, OH (US); Sandeep Gupta, Concord, OH (US); Aimee Dolores Reed, Willoughby, OH (US); Debasis Ghosh, Perrysburg, OH (US)

Correspondence Address: FAY SHARPE LLP, 1100 SUPERIOR AVENUE, SEVENTH FLOOR, CLEVELAND, OH, 44114, US

(21) Appl. No.: 11/244,614

(22) Filed: Oct. 6, 2005

(51) Int. Cl. A61K 31/519; C07D 487/02

(52) U.S. Cl. 514/264.1; 544/250; 544/279; 514/267

(57) ABSTRACT

Disclosed herein are pyrido[2,3-d]pyrimidines having the structure 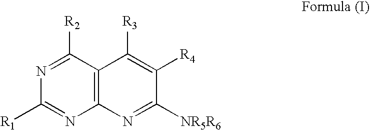
wherein R₁, and R₂ are independently selected from nitro and amino;
wherein R₃ is selected from alkyl (C1-C10) and cycloalkyl (C3-C8);
wherein R₄ is selected from hydrogen, benzyl, alkyl (C1-C10), cycloalkyl (C3-C8), arylalkyl (C4-C8), and aryl (C3-C8);
wherein R₅ and R₆ are independently selected from hydrogen, benzyl, alkyl (C1-C10), cycloalkyl (C3-C8), arylalkyl (C4-C14), and aryl (C3-C8);
wherein R₃ and R₄ may form a ring;
wherein R₅ and R₆ may form a ring or a heterocyclic structure; and
wherein R₅ and R₆ are independently unsubstituted or substituted, wherein each substituent is independently selected from halogen, amino, alkyl (C1-C6), haloalkyl (C1-C6), alkoxy(C1-C6), alkylenedioxy, aryl, heteroaryl, and cycloalkyl (C3-C8). Also disclosed are methods for the preparation of compounds of Formula I and various intermediates. These compounds are useful as NAD+-dependent DNA ligase inhibitors.

### BACKGROUND

This disclosure relates generally to the field of pharmaceutical chemistry. More particularly, the disclosure relates to the discovery of a group of pyrido[2,3-d]pyrimidines (also known as 5-deazapteridines) useful as NAD+-dependent DNA ligase inhibitors. Various embodiments of these compounds are disclosed. These compounds can be used in pharmaceutical compositions useful for the treatment of bacterial infections in mammals or for controlling the growth of disease-causing bacteria on surfaces requiring disinfection.

Bacterial infections remain one of the leading causes of death worldwide. Staphylococcus aureus, Streptococcus pneumnoniae, Escherichia coli, Enterococcus faecalis, Haemophilus influenzae, Moraxella catarrhalis, and Streptococcus pyogenes are among the major pathogens causing severe infections, which include otitis, sinusitis, pharyngitis, bronchitis, pneumonia, endocarditis, septicemia, and skin and urinary tract infections.

In inhibiting bacterial growth, it is desired to minimize side effects in mammals, especially human beings. This can be done by targeting portions of bacterial systems which are not shared by mammals. One such target is DNA ligase.

DNA is susceptible to damage caused by replication errors and by environmental factors such as radiation, oxidants, and alkylating agents. The repair and replication pathways converge on a common final step in which the continuity of the repaired DNA strand or the replicated lagging strand is restored by DNA ligase, an enzyme that converts nicks into phosphodiester bonds. Nicks are potentially deleterious lesions that, if not corrected, may give rise to double-strand breaks which are themselves overtly catastrophic if not repaired by homologous recombination or ligase-mediated non-homologous end-joining. Accordingly, a complete loss of DNA ligase function is lethal in the organisms tested.

DNA ligases are grouped into two families, ATP-dependent ligases and NAD+-dependent ligases, according to the cofactor required for ligase-adenylate formation. The NAD+-dependent DNA ligases have been described only in eubacteria. Genes encoding NAD+-dependent DNA ligases have been identified and sequenced from many eubacterial species. Every bacterial species encodes at least one NAD+-dependent DNA ligase and it is essential for the growth of E. coli and S. aureus. It is reasonable to believe that NAD+-dependent DNA ligase is essential for growth of bacteria. Moreover, no NAD+-dependent DNA ligase activity has been identified from a eukaryotic cellular source.

NAD+-dependent DNA ligases are therefore attractive targets for drug discovery. NAD+-dependent ligases are present in bacteria and are essential for bacterial growth. Moreover, they are structurally conserved among bacteria, but display unique substrate specificity and domain architecture compared to ATP-dependent DNA ligases. Therefore, inhibitors of bacterial NAD+-dependent DNA ligases would be outstanding candidates for effective broad spectrum antibiotic therapy, etc.

Pyrido[2,3-d]pyrimidines are known to have medicinal properties. See, for example, U.S. Pat. Nos. 4,512,992; 4,621,082; 5,508,281; 5,532,370; 5,654,307; 5,952,342; 6,750,241; and 6,787,341; these patents are hereby incorporated by reference in their entirety. Because their chemical structure is similar to that of known dihydrofolate reductase (DHFR) inhibitors, it is hypothesized that pyrido[2,3-d]pyrimidines might also be used to target DHFR. However, two specific DHFR inhibitors, methotrexate and trimethoprim, have been shown not to inhibit NAD+-dependent DNA ligase.

There is still a need for antibacterial compounds, especially compounds which will inhibit NAD+-dependent DNA ligase.

### BRIEF DESCRIPTION

Disclosed herein are pyrido[2,3-d]pyrimidine compounds having antibacterial properties. Also disclosed are pharmaceutical compositions comprising those compounds. Methods for producing the compounds are further disclosed. Methods for using the compounds to treat bacterial infections, as well as for their use in mammals, especially human beings, are also disclosed.

These and other non-limiting features of the compounds and methods of the present disclosure are more particularly described below.

### DETAILED DESCRIPTION

The present disclosure relates to novel compounds of Formula (I): 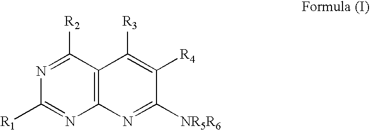
wherein R₁ and R₂ are independently selected from nitro and amino;
wherein R₃ is selected from alkyl (C1-C10) and cycloalkyl (C3-C8);
wherein R₄ is selected from hydrogen, alkyl (C1-C10), cycloalkyl (C3-C8), arylalkyl (C4-C8), and aryl (C3-C8);
wherein R₅ and R₆ are independently selected from hydrogen, alkyl (C1-C10), cycloalkyl (C3-C8), arylalkyl (C4-C14), and aryl (C3-C8);
wherein R₃ and R₄ may form a ring;
wherein R₅ and R₆ may form a ring or a heterocyclic structure; and
wherein R₅ and R₆ are independently unsubstituted or substituted, wherein each substituent is independently selected from halogen, amino, alkyl (C1-C6), haloalkyl (C1-C6), alkoxy(C1-C6), alkylenedioxy, aryl, heteroaryl, and cycloalkyl (C3-C8).

Additionally, also disclosed are methods for the preparation of compounds of Formula I and various intermediates. These compounds are useful as NAD+-dependent DNA ligase inhibitors. They can be used in pharmaceutical compositions useful for the treatment of bacterial infections in patients or for controlling the growth of disease-causing bacteria on surfaces requiring disinfection.

Compounds of Formula (I) have been found to be effective as antibacterial agents. In particular, these compounds have a low Minimum Inhibitory Concentration (MIC), a low IC₅₀, and a high LC₅₀. The MIC measures the concentration at which bacterial growth is inhibited; generally, a lower MIC is better. The IC₅₀ is the “inhibition concentration” that is required for 50% inhibition of an enzyme. Again, a lower IC₅₀ is generally better. The LC₅₀ is the dosage at which 50% of test subjects die after taking the compound. A higher LC₅₀ is better.

In further embodiments, the compound has the structure of Formula (II): 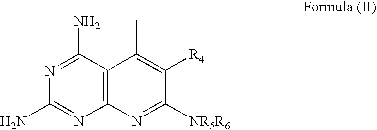
wherein R₄ is methyl, n-butyl, isopropyl, benzyl, or cyclopentyl; wherein R₅ is hydrogen or methyl; and wherein R₆ is benzyl, substituted benzyl, or cyclohexyl.

Specific embodiments of Formula (II) include the following compounds: 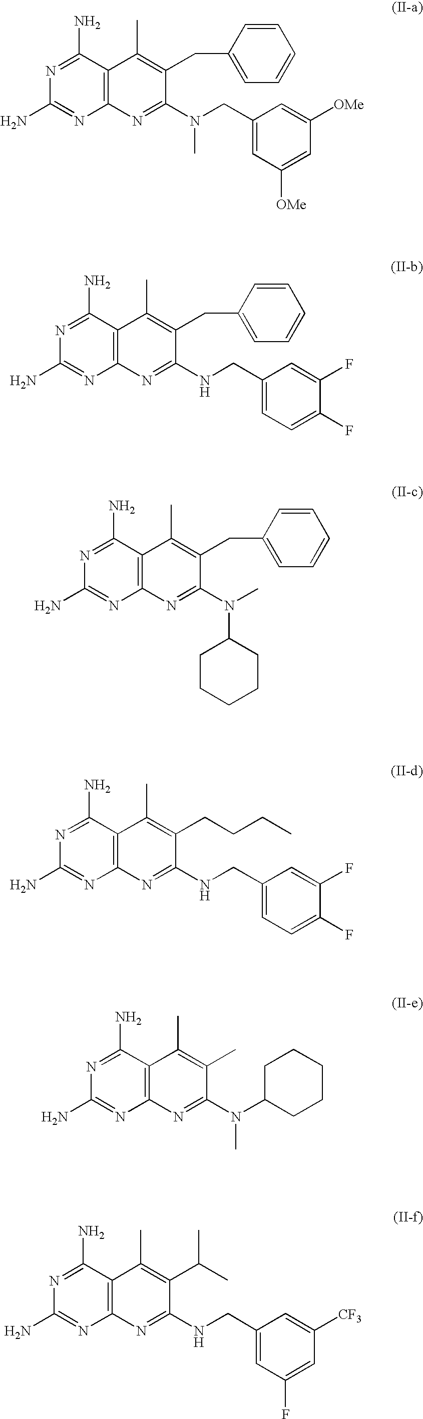 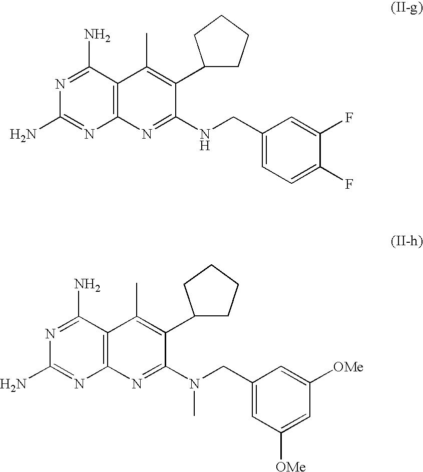
The embodiments of Formula (II) wherein R₃ is methyl and R₄ is benzyl are especially effective as antibacterial agents; These three embodiments are (II-a), (II-b), and (II-c). The embodiment of (II-d) is also especially effective.

In further embodiments, the compound has the structure of Formula (III):

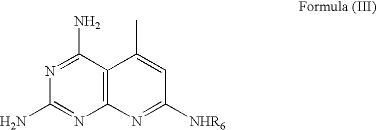

Specific embodiments of Formula (III) include the following compounds: 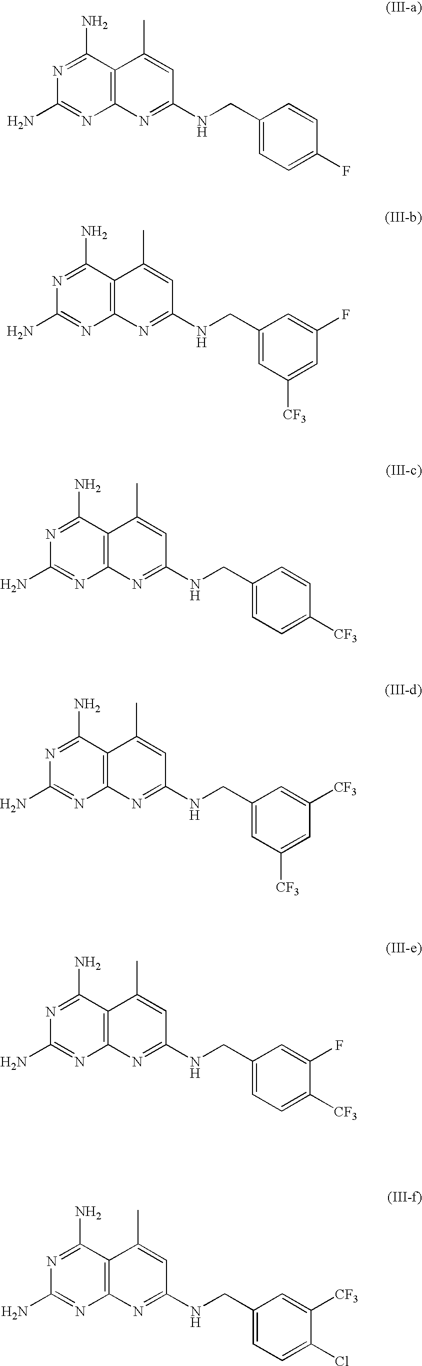 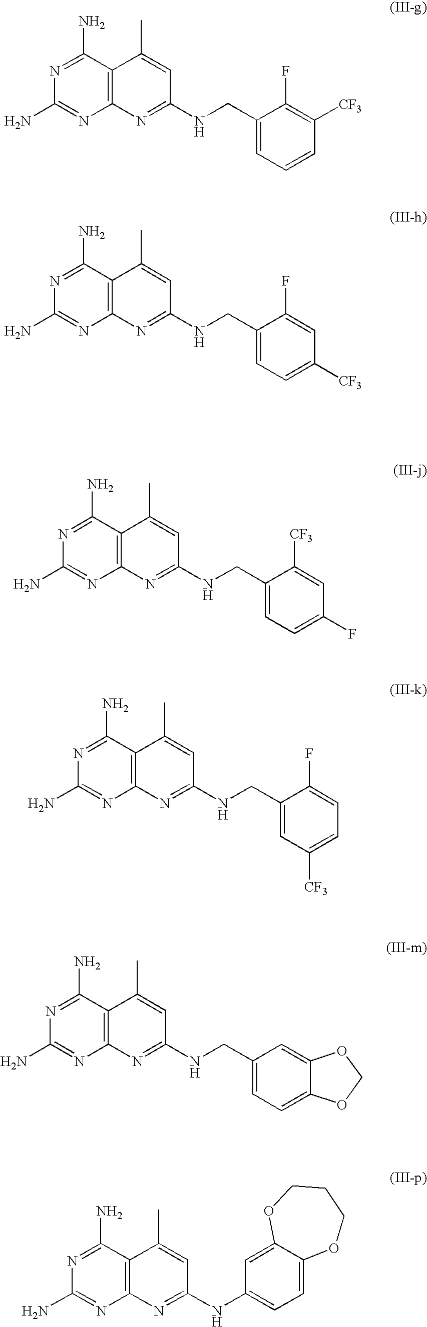
The embodiment (III-b) is especially effective as an antibacterial agent.

In further embodiments, the compound has the structure of Formula (IV): 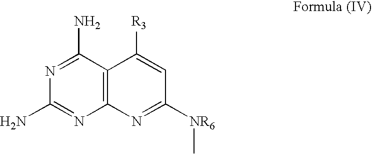
wherein R₃ is alkyl; and wherein R₆ is cyclohexyl, benzyl or substituted benzyl. In specific embodiments, R₃ is methyl or n-propyl.

Specific embodiments of Formula (IV) include the following compounds:

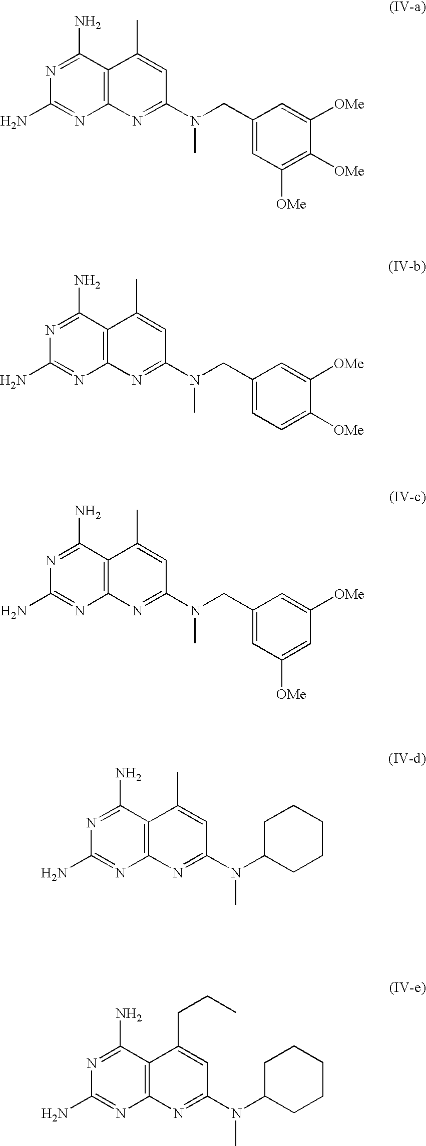

In further embodiments, the compound has the structure of Formula (V): 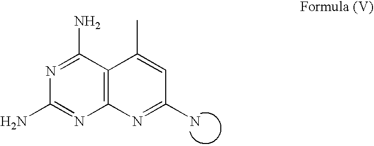
Here, R₅ and R₆ form a ring or a heterocyclic structure.

Specific embodiments of Formula (V) include the following compounds:

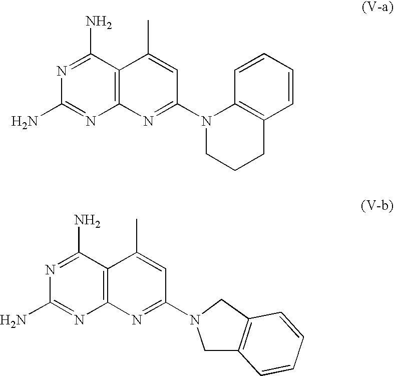

In further embodiments, the compound has the structure of Formula (VI):

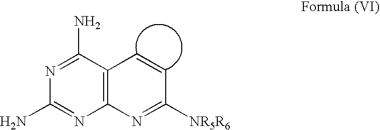

wherein R₃ and R₄ form a ring. In further embodiments, the compound of Formula (VI) has the structure of Formula (VII):

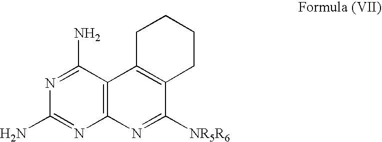

Specific embodiments of Formula (VII) include the following compounds:

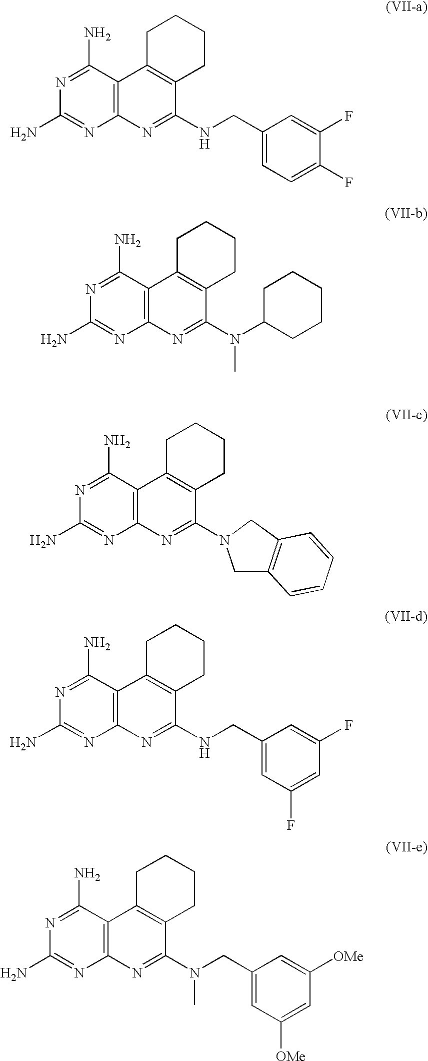

In further embodiments, the compound has the structure of Formula (VIII): 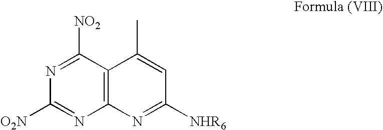
wherein R₆ is benzyl or substituted benzyl.

Specific embodiments of Formula (VIII) include the following compounds:

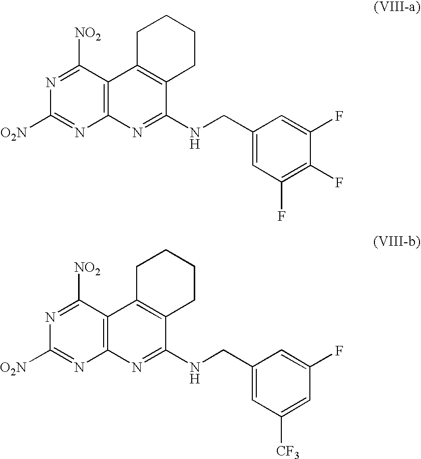

Generic compounds of Formula (I) share several features. First, R₃ is a small hydrophobic residue. This was found to lead to compounds having low MICs. In contrast, a large lipophilic residue at R₃ was found not to reduce DNA ligase activity. Large hydrophobic substituents at R₅ or R₆ were found to lead to good MICs and IC₅₀s.

As can be seen in the specific embodiments above, several specific groups at R₅ or R₆ were found to give good antibacterial activity to the pyrido[2,3-d]pyrimidine. Those specific groups include cyclohexyl, dimethoxybenzyl, difluorobenzyl, and fluoro-trifluoromethylbenzyl. The heterocyclic structure 3,4-benzopyrrolidino was also found to give good antibacterial activity.

Compounds of Formula (I) can be prepared by various methods. For example, the specific embodiments of Formula (I) can be prepared using the three-step method described below.

First, a 2,4,6-triaminopyrimidine (1) is reacted with an acetoacetate (2) to form a 2,4-diamino-7,8-dihydro-7-oxo-pyrido[2,3-d]pyrimidine (3).

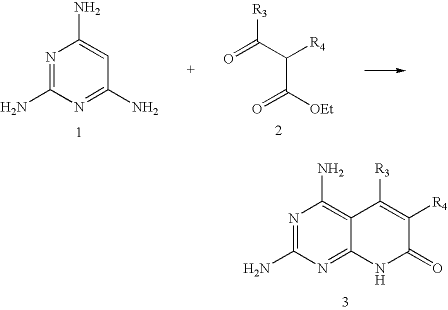

Next, the 7-oxo site is chlorinated to yield a 2,4-diamino-7-chloro-pyrido[2,3-d]pyrimidine (4).

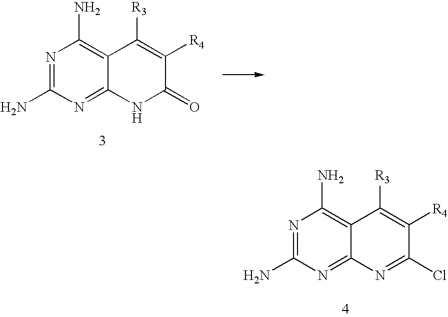

Finally, the 2,4-diamino-7-chloro-pyrido[2,3-d]pyrimidine (4) is reacted with a disubstituted amine to give the compound of Formula (I) (6).

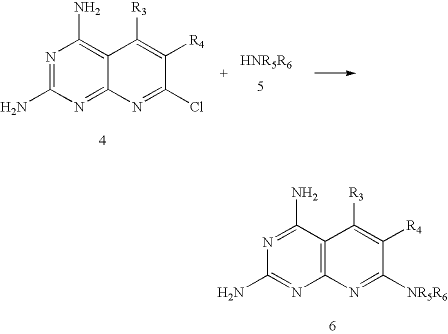

The amino groups at the 2 and 4 position can be oxidized to form nitro groups using methods known in the prior art. Indeed, 2,4-dinitropyrido[2,3-d]pyrimidines are generally formed as byproducts of the amination reaction. 2,4-diaminopyrido[2,3-d]pyrimidines and 2,4-dinitropyrido[2,3-d]pyrimidines can be separated using conventional methods.

With regard to the chlorination of the 7-oxo site, the art teaches at least one method of chlorination. For example, the Vilsmeier dimethylformamide-thionyl chloride reagent may be used. See Grivsky et al., J. Med. Chem. 1980, vol. 23, pgs. 327-329, the entirety of which is hereby incorporated by reference. Grivsky also reported that the use of phosphorus reagents led to complexes which could not be successfully reduced. However, here a phosphorus reagent was successfully used to obtain pyrido[2,3-d]pyrimidines of the present disclosure in good yield. It is expected that a phosphorous reagent can thus be used to chlorinate any such oxo site on a pyrido[2,3-d]pyrimidine.

The chlorination using a phosphorus reagent may be performed by suspending the oxopyrido[2,3-d]pyrimidine in the phosphorus reagent to form a reaction mixture. The reaction mixture is heated, then cooled and adjusted to a basic pH. It is then purified to obtain a chloropyrido[2,3-d]pyrimidine. The phosphorus reagent may be selected from the group consisting of POCl₃, PCl₅, PCl₃, and CH₃Cl₂OP. The reaction mixture is heated to a temperature of from about 80 to about 150° C. for a period of from about 2 to 12 hours. The reaction mixture is stirred while it is being heated. The reaction mixture may be heated under an inert gas, such as argon or nitrogen, to prevent any possible oxidation. The reaction mixture is cooled to a temperature of from about 25 to about 0° C. over a period of from about 0.5 to about 2 hours. If desired, the reaction mixture can be cooled by quenching it. The reaction mixture is then adjusted to a basic pH of from about 8 to about 12, and in specific embodiments to a pH of about 10.

In a specific embodiment, the chlorination is performed by suspending the intermediate 7-oxopyrido[2,3-d]pyrimidine (3) in neat POCl₃ and stirring and heating the mixture under argon for a period of about 2 hours. The excess POCl₃ is then quenched with ice and the pH is adjusted to a basic pH of about 10.

The compounds of the present disclosure can be used as such, be administered in the form of pharmaceutically acceptable salts derived from inorganic or organic acids, or used in combination with one or more pharmaceutically acceptable excipients. The phrase “pharmaceutically acceptable salt” means those salts which are, within the scope of sound medical judgment, suitable for use in contact with the tissues without undue toxicity, irritation, allergic response, and the like, and are commensurate with a reasonable benefit/risk ratio. Pharmaceutically acceptable salts are well known in the art. The salts can be prepared either in situ during the final isolation and purification of the compounds of the invention or separately by reacting the acidic or basic drug substance with a suitable base or acid respectively. Typical salts derived from organic or inorganic acids salts include, but are not limited to hydrochloride, hydrobromide, hydroiodide, acetate, adipate, alginate, citrate, aspartate, benzoate, bisulfate, gluconate, fumarate, hydroiodide, lactate, maleate, oxalate, palmitoate, pectinate, succinate, tartrate, phosphate, glutamate, and bicarbonate. Typical salts derived from organic or inorganic bases include, but are not limited to lithium, sodium, potassium, calcium, magnesium, ammonium, monoalkylammonium such as meglumine, dialkylammonium, trialkylammonium, and tetralkylammonium.

The mode of administration of the pharmaceutical compositions can be oral, rectal, intravenous, intramuscular, intracisternal, intravaginal, intraperitoneal, bucal, subcutaneous, intrasternal, nasal, or topical. The compositions can also be delivered at the target site through a catheter, an intracoronary stent (a tubular device composed of a fine wire mesh), a biodegradable polymer, or biological carriers including, but not limited to, antibodies, biotin-avidin complexes, and the like. Dosage forms for topical administration of a compound of this invention include powders, sprays, ointments and inhalants. The active compound is mixed under sterile conditions with a pharmaceutically acceptable carrier and any needed preservatives, buffers or propellants. Opthalmic formulations, eye ointments, powders and solutions are also contemplated as being within the scope of this disclosure.

Actual dosage levels of active ingredients and the mode of administration of the pharmaceutical compositions of this disclosure can be varied in order to achieve the effective therapeutic response for a particular patient. The phrase “therapeutically effective amount” means a sufficient amount of the compound to treat disorders, at a reasonable benefit/risk ratio applicable to any medical treatment. It will be understood, however, that the total daily usage of the compounds and compositions of the present disclosure will be decided by the attending physician within the scope of sound medical judgment. The total daily dose of the compounds of this disclosure may range from about 0.0001 to about 1000 mg/kg/day. For purposes of oral administration, more preferable doses can be in the range from about 0.001 to about 5 mg/kg/day. If desired, the effective daily dose can be divided into multiple doses for purposes of administration; consequently, single dose compositions may contain such amounts or submultiples thereof to make up the daily dose. The specific therapeutically effective dose level for any particular patient will depend upon a variety of factors including the disorder being treated and the severity of the disorder; medical history of the patient, activity of the specific compound employed; the specific composition employed, age, body weight, general health, sex and diet of the patient, the time of administration, route of administration, the duration of the treatment, rate of excretion of the specific compound employed, drugs used in combination or coincidental with the specific compound employed; and the like.

The compounds of the present disclosure can be formulated together with one or more non-toxic pharmaceutically acceptable diluents, carriers, adjuvants, and antibacterial and antifungal agents such as parabens, chlorobutanol, phenol, sorbic acid, and the like. Proper fluidity can be maintained, for example, by the use of coating materials such as lecithin, by the maintenance of the required particle size in the case of dispersions, and by the use of surfactants. In some cases, in order to prolong the effect of the drug, it is desirable to decrease the rate of absorption of the drug from subcutaneous or intramuscular injection. This can be accomplished by suspending crystalline or amorphous drug substance in a vehicle having poor water solubility such as oils. The rate of absorption of the drug then depends upon its rate of dissolution, which, in turn, may depend upon crystal size and crystalline form. Prolonged absorption of an injectable pharmaceutical form can be achieved by the use of absorption delaying agents such as aluminum monostearate or gelatin.

The compounds of the present disclosure can be administered enterally or parenterally in solid or liquid forms. Compositions suitable for parenteral injection may comprise physiologically acceptable, isotonic sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, or mixtures of these substances.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters), and poly(anhydrides). Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present disclosure can also be administered in the form of liposomes. Methods to form liposomes are known in the art. As is known in the art, liposomes are generally derived from phospholipids or other lipid substances. Liposomes are formed by mono- or multi-lamellar hydrated liquid crystals, which are dispersed in an aqueous medium. Any non-toxic, physiologically acceptable and metabolizable lipid capable of forming liposomes can be used. The present compositions in liposome form can contain, in addition to a compound of the present disclosure, stabilizers, preservatives, excipients and the like. The preferred lipids are natural and synthetic phospholipids and phosphatidyl cholines (lecithins).

The compounds of the present disclosure can also be administered in the form of a ‘prodrug’ wherein the active pharmaceutical ingredients, represented by Formulas 1-3, are released in vivo upon contact with hydrolytic enzymes such as esterases and phophatases in the body. The term “pharmaceutically acceptable prodrugs” as used herein represents those prodrugs of the compounds of the present disclosure which are, within the scope of sound medical judgment, suitable for use in contact with the tissues without undue toxicity, irritation, allergic response, and the like, commensurate with a reasonable benefit/risk ratio, and effective for their intended use. A thorough discussion is provided in T. Higuchi and V. Stella [Higuchi, T. and Stella, V. Pro-drugs as Novel Delivery Systems, V. 14 of the A.C.S. Symposium Series; Edward B. Roche, Ed., Bioreversible Carriers in Drug Design 1987, American Pharmaceutical Association and Pergamon Press], which is incorporated herein by reference.

Although the disclosure has been described with respect to specific embodiments, it is not intended to be limited thereto. Those skilled in the art will recognize that variations and modifications including equivalents, substantial equivalents, similar equivalents and the like may be made therein which are within the spirit of the disclosure and the scope of the claims. The development of the present disclosure will further be illustrated in the following non-limiting working examples, it being understood that these examples are intended to be illustrative only and that the disclosure is not intended to be limited to the materials, conditions, process parameters and the like recited herein.

### EXAMPLE 1

2,4-diamino-5-methyl-7-[(3-fluoro-5-trifluoromethylbenzyl)amino]-pyrido[2,3-d]pyrimidine, shown in Formula (III-b) above, was synthesized.

To a stirred suspension of 2,4,6-triaminopyrimidine (1) (15 g, 120 mmol) in diphenyl ether (180 ml) was charged ethyl acetoacetate (7) (15.6 g, 120 mmol), and the reaction mixture was heated at 210°° C. for 3 hrs. During heating, 14 ml of low boilers (EtOH and water) were collected. The reaction mixture was cooled to room temperature and slurried with 180 ml of methanol. The solid was filtered out and suspended in 1500 ml of boiling water. It was filtered out and washed with 750 ml of boiling water. The pale-yellow solid was re-suspended in 750 ml of methanol, filtered off and dried in vacuum overnight. Yield: 11.4 g (49.8%) of 2,4-diamino-5-methyl-8H-pyrido[2,3-d]pyrimidin-7-one (8).

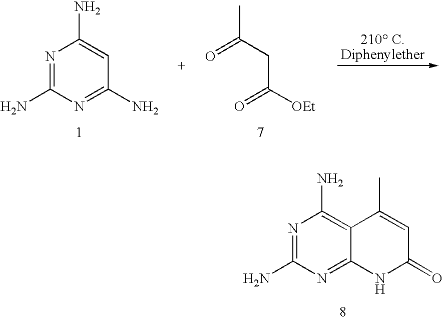

To a stirred suspension of 2,4-diamino-5-methyl-8H-pyrido[2,3-d]pyrimidin-7-one (8) (27 g, 140 mmol) in anhydrous chloroform (550 ml) was added DMF (103.3, 1400 mmol) followed by thionyl chloride (167.7 g, 1400 mmol) slowly at 0° C. under nitrogen. After complete addition, the reaction mixture was heated to reflux and maintained for 1.5 hours. It was cooled to room temperature and concentrated on rotavapor at 50° C. The viscous residue was dissolved in 50% ethanol-water (1500 ml). The solution was cooled to 0° C. and basified to pH 10 with 2M sodium hydroxide solution. The yellow solid was filtered off and dissolved in a solution of 10% methanol in chloroform (3.5 L). The solution was filtered and evaporated to dry. The solid was re-suspended in dioxane-water (9:1) and the mixture was basified with 5 M sodium hydroxide solution to pH 10. The solution was stirred at room temperature overnight. The mixture was concentrated on rotavapor. The solid was filtered out, washed with water and dried in vacuum to give pale-yellow solid 10.2 g (34.5%) of 2,4-diamino-5-methyl-7-chloro-pyrido[2,3-d]pyrimidine (9).

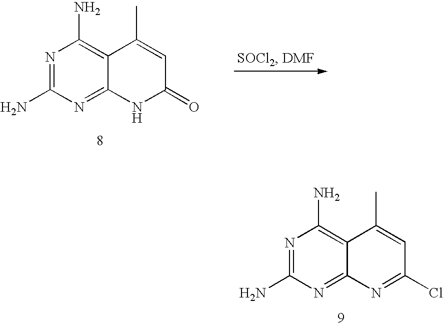

2,4-diamino-5-methyl-7-chloro-pyrido[2,3-d]pyrimidine (9) (0.1 g, 0.5 mmol) and (3-fluoro-5-trifluoromethylbenzyl)amine (10) (0.22 ml, 1.5 mmol) in dimethylsulfoxide (DMSO) (2 ml) were heated at 120° C. under argon overnight. The reaction mixture was concentrated with rotavapor. Ethyl ether (30 ml) was added. The precipitate was collected and suspended in water (10 ml) with stirring. To the suspension was added saturated sodium carbonate solution (1 ml). The undissolved solid was collected, washed with ethyl ether and dried. The crude product was separated by prep-HPLC to give major product (15 mg) of 2,4-diamino-5-methyl-7-[(3-fluoro-5-trifluoromethylbenzyl)amino]-pyrido[2,3-d]pyrimidine (11) and by-product (4.2 mg) of 2,4-dinitro-5-methyl-7-[(3-fluoro-5-trifluoromethylbenzyl)amino]-pyrido[2,3-d]pyrimidine (12). The major product (11) corresponded to the compound of Formula (III-b).

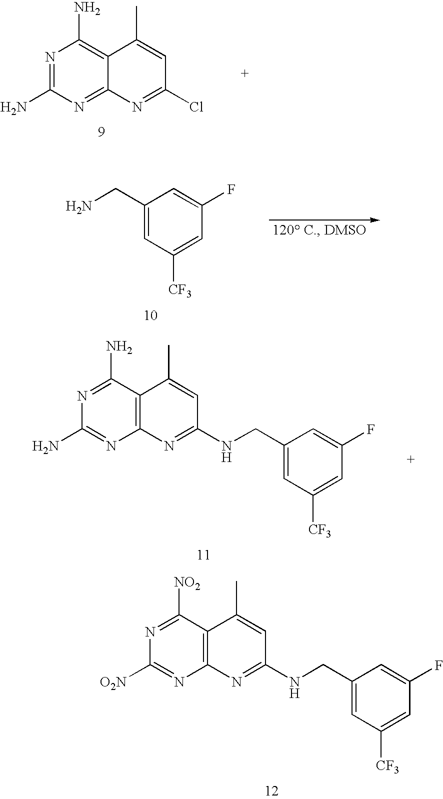

### EXAMPLE 2

2,4-diamino-5-methyl-6-benzyl-7-[(3,5-dimethoxybenzyl)methylamino]-pyrido[2,3-d]pyrimidine, shown in Formula (II-a) above, was synthesized.

A mixture of 2,4,6-triaminopyrimidine (1) (1.0 eq., 10 mmol) and ethyl 2-benzylacetoacetate (13) (1.2 eq.) in diphenyl ether (3 mL/mmol) was stirred at 210° C. for 2 hours under argon. A second portion of ethyl 2-benzylacetoacetate (1.2 eq.) was added at near room temperature and heated at 210° C. for another 2 hours under argon. The mixture was cooled down to room temperature and 30 mL MeOH was added. The resulting precipitates were filtered and washed with MeOH twice. The precipitates were further suspended in boiling water (30 mL) and stirred for 30 min. and washed with hot water, followed by MeOH, dried in vacuo to obtain 2,4-diamino-5-methyl-6-benzyl-8H-pyrido[2,3-d]pyrimidin-7-one (14) (yield 81%-85%).

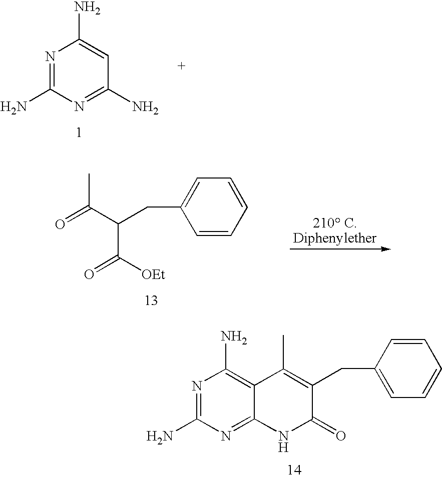

The 2,4-diamino-5-methyl-6-benzyl-8H-pyrido[2,3-d]pyrimidin-7-one (14) was suspended in neat POCl₃ and the mixture was stirred and heated at 110° C. under Argon for 2 hours. The excess POCl₃ was removed on a rotavapor and the residue was chilled in an ice bath and carefully quenched with ice, then adjusted to pH 10 with 10% NaOH. The precipitates formed were collected and washed with water. The resulting residue was further wash/extracted with 10% MeOH in dichloromethane. The remaining residue was discarded and the organic solvent was concentrated to obtain 2,4-diamino-5-methyl-6-benzyl-7-chloro-pyrido[2,3-d]pyrimidine (15) together with residual 2,4-diamino-5-methyl-6-benzyl-8H-pyrido[2,3-d]pyrimidin-7-one (14). The 2,4-diamino-5-methyl-6-benzyl-7-chloro-pyrido[2,3-d]pyrimidine (15) was used directly in the next step without further purification.

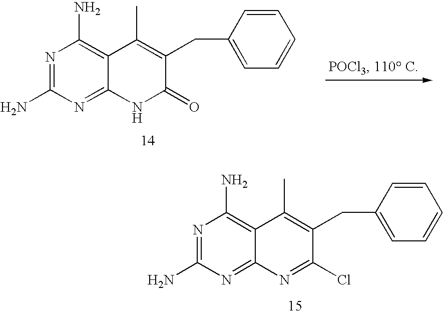

2,4-diamino-5-methyl-6-benzyl-7-chloro-pyrido[2,3-d]pyrimidine (15) (0.277 mmol) was suspended in 1 mL DMSO and (3,5-dimethoxybenzyl)methylamine (16) (3.0 eq.) was added to the above solution. The resulting mixture was placed under argon atmosphere and stirred at 120° C. overnight. The reaction mixture was cooled to room temperature and the DMSO was removed at 70-80° C. under high vacuum. The resulting residue was cooled to room temperature. The residue was washed by adding ether (20 mL) and then decanting the ether; the wash was then repeated. 10 mL water and 1 mL saturated aqueous sodium carbonate were added. After sonicating, nice yellow precipitates were formed. The precipitates were filtered, washed with water till the pH was neutral and washed with ether again to remove any residual amine (16). The final compound, 2,4-diamino-5-methyl-6-benzyl-7-[(3,5-dimethoxybenzyl)methylamino]-pyrido[2,3-d]pyrimidine (17), was obtained after drying under high vacuum.

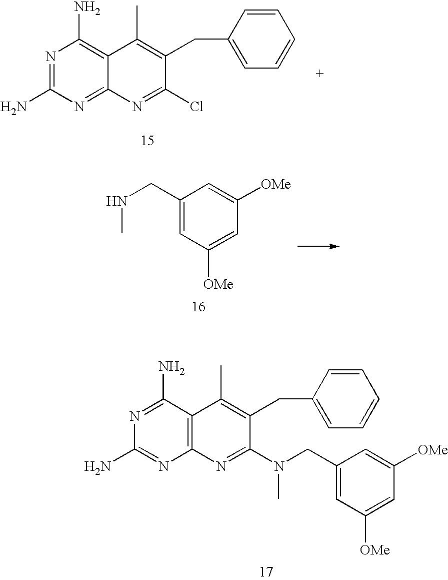

### EXAMPLE 3

The compounds of Formulas 1-VIII were evaluated as inhibitors of NAD+-dependent DNA ligase inhibitors. Their antimicrobial activity was evaluated against bacteria using the broth microdilution method as described by Clinical Laboratory Standards Institute (formerly NCCLS). Activity was assessed against the following bacterial strains: Staphylococcus aureus ATCC 29213, Streptococcus pneumoniae ATCC 49619, Escherichia coli ATCC 25922, E. coli CGSC 5633 (ToIC mutant), Haemophilus influenzae ATCC 49766, Streptococcus pyogenes ATCC 19615, Moraxella catterhalis ATCC 25238, Enterococcus faecalis ATCC 29212. The lowest concentration of compound which inhibited visual growth of each bacterium, the minimum inhibitory concentration (MIC), was determined.

Acute toxicity was assessed against two mammalian cell lines, HepG2 (human hepatocyte) and NIH 3T3 (mouse fibroblast). Cell viability was measured by an in vitro assay using Alamar blue. The concentration of compound which inhibited 50% of the cell growth (LC₅₀) was determined.

Activity against DNA ligase enzyme was measured using purified enzyme from S. pneumoniae. An AMP release assay was used to assess activity of compounds against the enzyme. AMP, released as a result of the ligation of oligonucleotides, is converted to ATP, which is then measured using luciferase enzyme. The concentration of compound which inhibited 50% of the ligation activity of the enzyme (IC₅₀) was determined.

The evaluations of these compounds are set forth below in Table 1.

TABLE 1

| | | MIC | | MIC |
|---|---|---|---|---|
| | | S. aureus | | E. coli |
| Formula | | ATCC | MIC | ATCC |
| Number | Structure | 29213 | S. pneumoniae | 25922 |
| II-a | 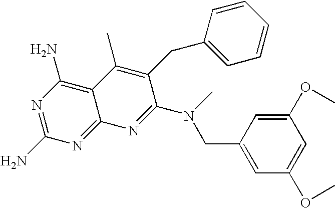 | 4 | 0.125 | >128 |
| II-b | 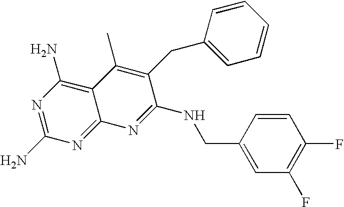 | 4 | 4 | 32 |
| II-c | 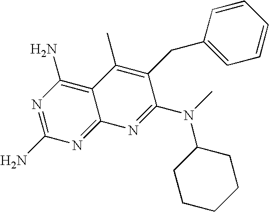 | 4 | 4 | 128 |
| II-d | 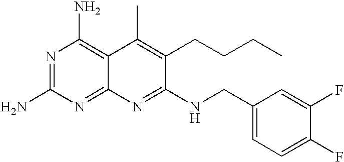 | 4 | 1 | 32 |
| II-e | 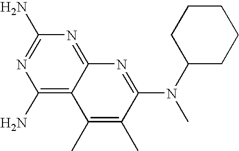 | 32 | 4 | 64 |
| II-f | 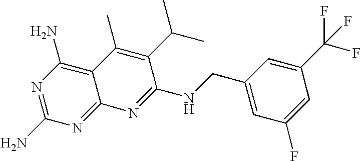 | 8 | 8 | 128 |
| II-g | 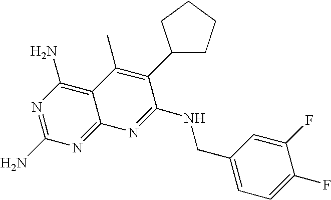 | 4 | 4 | >128 |
| II-h | 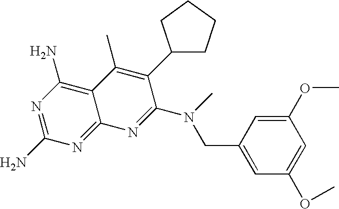 | 4 | 0.5 | 32 |
| III-a | 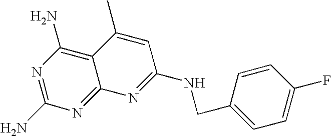 | 32 | 64 | 32 |
| III-b | 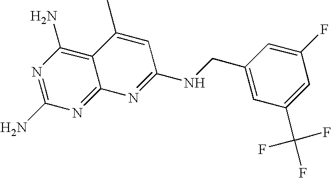 | 16 | 1 | 16 |
| III-c | 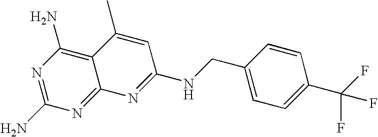 | 16 | 4 | 16 |
| III-d | 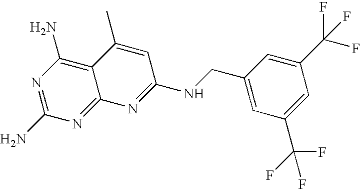 | 16 | 2 | 16 |
| III-f | 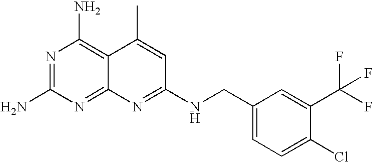 | 8 | 1 | 16 |
| III-g | 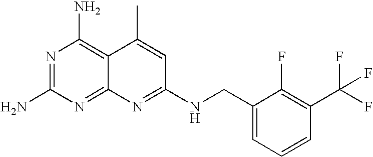 | 16 | 2 | 16 |
| III-h | 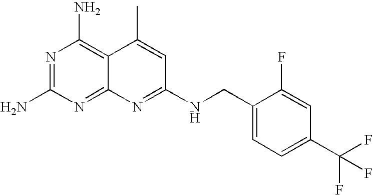 | 16 | 4 | 16 |
| III-j | 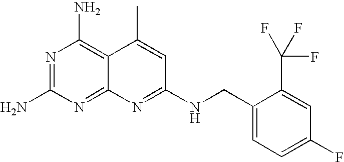 | 16 | 1 | 32 |
| III-k | 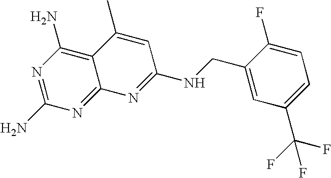 | 16 | 2 | 32 |
| IV-a | 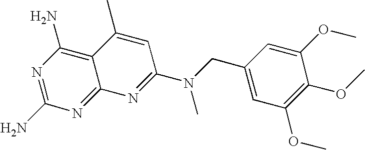 | 64 | 4 | 15 |
| IV-b | 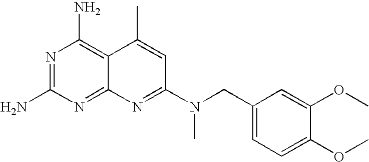 | 64 | 2 | 64 |
| IV-c | 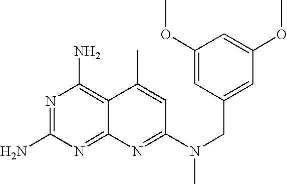 | 32 | 0.5 | 16 |
| IV-d | 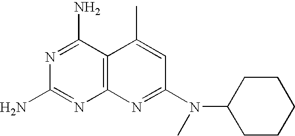 | 64 | 8 | 64 |
| IV-e | 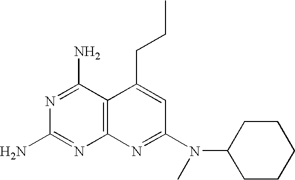 | 32 | 2 | 128 |
| V-a | 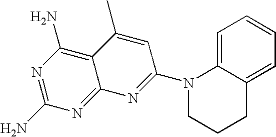 | 32 | 8 | 64 |
| V-b | 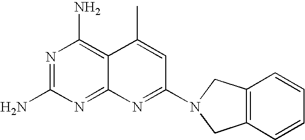 | 16 | 8 | 64 |
| III-m | 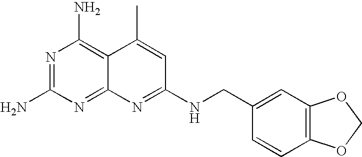 | 64 | 2 | 64 |
| III-p | 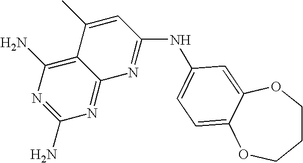 | 128 | 4 | 64 |
| VII-a | 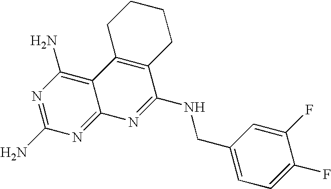 | 16 | 4 | >128 |
| VII-b | 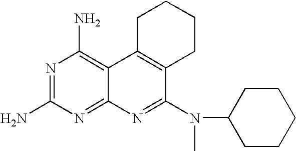 | 8 | 8 | 128 |
| VII-c | 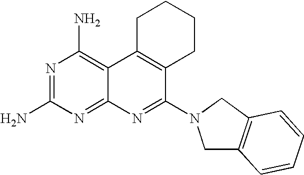 | 16 | 8 | >128 |
| VII-d | 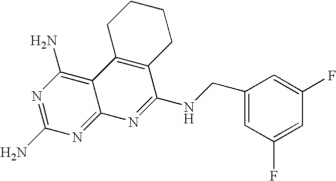 | 32 | 8 | 64 |
| VII-e | 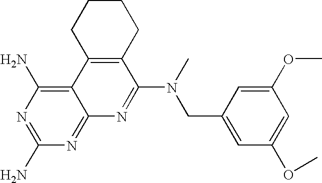 | 32 | 0.5 | 64 |
| VIII-a | 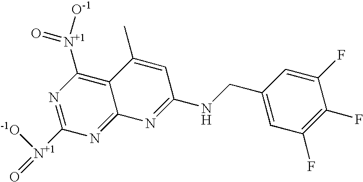 | 8 | 16 | 64 |
| VIII-b | 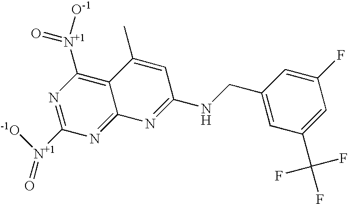 | 8 | 8 | 64 |

| | | | | | | Acute | Acute | |
|---|---|---|---|---|---|---|---|---|
| | MIC | | | | | Tox | Tox | Ligase |
| | E. coli | | | | | LC50 | LC50 | activity |
| Formula | CGSC | MIC | MIC | MIC | MIC | μM/ | μM/ | IC50 |
| Number | 5633 | E. faecalis | H. influenzae | M. catarrhalis | S. pyogenes | HepG2 | NIH-3T3 | μM |
| II-a | <0.125 | <0.125 | >128 | 4 | 4 | 12.81 | 5.57 | |
| II-b | 0.125 | 1 | 64 | 2 | 1 | 14 | 2.94 | 12.64 |
| II-c | <0.125 | 2 | >128 | 2 | 2 | 15.01 | 5.13 | 11.27 |
| II-d | <0.125 | 2 | 16 | 2 | 2 | 17.2 | 5.16 | |
| II-e | 0.125 | 1 | | 4 to 8 | 2 to 8 | 19.03 | 10.91 | 79.1 |
| II-f | | 2 | 32 | 4 | 8 | | | |
| II-g | 2 | 4 | 16 | 1 | 4 | 16.45 | 2.67 | |
| II-h | <0.125 | <0.125 | 4 | 1 | 1 | 17.49 | 3.26 | |
| III-a | 0.25 | 4 | 2 | 8 | 2 | 24.03 | 8.14 | |
| III-b | <0.125 | 0.5 | 32 | 8 | 4 | 20.92 | 8.34 | |
| III-c | 0.25 | 4 | 16 | 8 | 4 | 22.77 | 11.2 | |
| III-d | 1 | 1 | 16 | 4 | 2 | 19.48 | 7.72 | |
| III-f | <0.125 | 1 | 16 | 2 | 2 | 15.19 | 8.32 | |
| III-g | 0.5 | 2 | 32 | 4 | 4 | | | |
| III-h | 0.5 | 4 | 16 | 4 | 4 | | | |
| III-j | 0.25 | 1 | 16 | 4 | 4 | | | |
| III-k | | 0.5 | 32 | 16 | 16 | | | |
| IV-a | <0.125 | 2 | 64 | 16 | 64 | | | 15.2 |
| IV-b | 0.125 | | 16 | 8 | | >28.22 | 13.25 | |
| IV-c | <0.125 | 0.5 | 32 | 1 | 8 | 37.38 | 14.57 | |
| IV-d | 0.125 | 2 | 51 | 16 | 32 | 23.42 | 12.75 | 15.2 |
| IV-e | 0.5 | 1 | | 4 | 1 | 15.1 | 9.9 | 80.6 |
| V-a | <0.125 | <0.125 | 16 | 16 | 32 | 27.09 | 11.52 | |
| V-b | 2 | 8 | >128 | 32 | 16 | 8.85 | 8.09 | 7.35 |
| III-m | 0.5 | 2 | 8 | 16 | 8 | | | |
| III-p | 0.125 | 0.5 | | 16 | | 20.01 | 15.61 | 11.4 |
| VII-a | 0.5 | 8 | 64 | 8 | 32 | 19.2 | 1.24 | 17.32 |
| VII-b | <0.125 | 4 | 64 | 4 | 4 | 19.19 | 1.37 | 5.94 |
| VII-c | | 1 | >128 | 4 | 16 | | | |
| VII-d | | 8 | 32 | 16 | 8 | | | |
| VII-e | 1 | <0.1250 | 16 | 4 | 2 | | | |
| VIII-a | 4 | 16 | 32 | 2 | 8 | >30.77 | 22.18 | |
| VIII-b | 2 | 4 | 64 | 4 | 4 | >16.420 | 12.1 | |

The above results demonstrate that the compounds of Formulas I-VIII are effective inhibitors of NAD+-dependent DNA ligase. With regard to MIC activity, the MICs needed to inhibit S. aureus and E. coli were especially considered. The compounds generally exhibited better activity against Gram-negative bacteria copared to Gram-positive bacteria.

Furthermore, as it will be appreciated by those skilled in the art, the present disclosure also relates to methods of using the compounds of Formulas I-VIII in a patient for therapeutic or prophylactic purposes. The disclosure also relates to the use of these compounds for controlling the growth of disease-causing bacteria on surfaces requiring disinfection.

The foregoing description is, at present, describes specific embodiments of the present disclosure. However, it is contemplated that various changes and modifications apparent to those skilled in the art may be made without departing from the present invention. Therefore, the foregoing description is intended to cover all such changes and modifications encompassed within the spirit and scope of the present disclosure, including all equivalent aspects.

1. A compound having the structure shown in Formula (I): 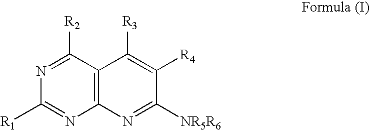
wherein R₁ and R₂ are independently selected from nitro and amino;
wherein R₃ is selected from alkyl (C1-C10) and cycloalkyl (C3-C8);
wherein R₄ is selected from hydrogen, benzyl, alkyl (C1-C10), cycloalkyl (C3-C8), arylalkyl (C4-C8), and aryl (C3-C8);
wherein R₅ and R₆ are independently selected from hydrogen, benzyl, alkyl (C1-C10), cycloalkyl (C3-C8), arylalkyl (C4-C14), and aryl (C3-C8);
wherein R₃ and R₄ may form a ring;
wherein R₅ and R₆ may form a ring or a heterocyclic structure; and
wherein R₅ and R₆ are independently unsubstituted or substituted, wherein each substituent is independently selected from halogen, amino, alkyl (C1-C6), haloalkyl (C1-C6), alkoxy(C1-C6), alkylenedioxy, aryl, heteroaryl, and cycloalkyl (C3-C8);
or a pharmaceutically acceptable salt thereof.

2. The compound of claim 1, having the structure shown in Formula (II): 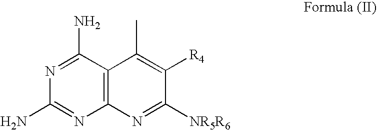
wherein R₄ is methyl, n-butyl, isopropyl, benzyl, or cyclopentyl;
wherein R₅ is hydrogen or methyl; and
wherein R₆ is benzyl, substituted benzyl, or cyclohexyl.

3. The compound of claim 1, having the structure shown in Formula (III):

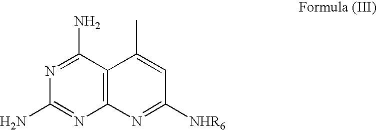

4. The compound of claim 1, having the structure shown in Formula (IV): 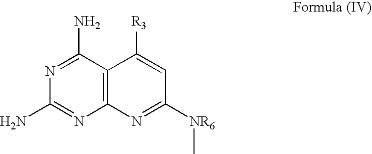
wherein R₃ is alkyl; and wherein R₆ is cyclohexyl, benzyl or substituted benzyl.

5. The compound of claim 1, having the structure shown in Formula (V): 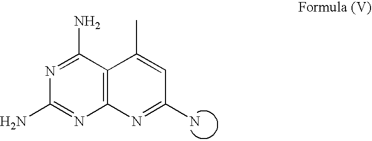
wherein R₅ and R₆ form a ring or a heterocyclic structure.

6. The compound of claim 5, wherein the heterocylic structure is 3,4-benzopyrrolidino.

7. The compound of claim 5, wherein the heterocylic structure is 1,2,3,4-tetrahydroquinolino.

8. The compound of claim 1, having the structure shown in Formula (VI): 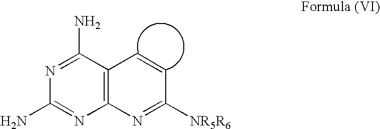
wherein R₃ and R₄ form a ring.

9. The compound of claim 8, having the structure shown in Formula (VII):

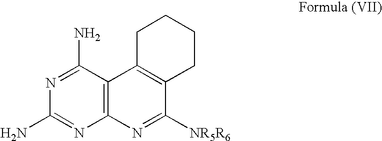

10. The compound of claim 1, having the structure shown in Formula (VIII): 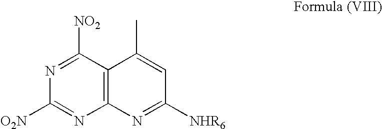
wherein R₆ is benzyl or substituted benzyl.

11. The compound of claim 1, wherein R₃ is methyl and R₄ is benzyl.

12. The compound of claim 1, wherein R₅ is methyl and R₆ is cyclohexyl.

13. The compound of claim 1, wherein R₅ and R₆ form a heterocyclic structure which is 3,4-benzopyrrolidino.

14. The compound of claim 1, wherein R₆ is dimethoxybenzyl.

15. The compound of claim 1, wherein R₆ is difluorobenzyl.

16. The compound of claim 1, wherein R₆ is fluoro-trifluoromethylbenzyl.

17. A pharmaceutical composition, comprising:
the compound of claim 1 or its pharmaceutically acceptable salt; and
a pharmaceutically acceptable buffer, carrier, diluent, or excipient.

18. The pharmaceutical composition of claim 17, further comprising an antifungal compound or an antibacterial compound other than the compound of claim 1 or its pharmaceutically acceptable salt.

19. A method of controlling the growth of a bacterium susceptible to the antibacterial activity of the compound of claim 1 or its pharmaceutically acceptable salt, comprising providing an therapeutically effective amount of the compound or its pharmaceutically acceptable salt to a locus where the bacterium is present.

20. A method of controlling the growth of a bacterium susceptible to the antibacterial activity of the compound of claim 1 or its pharmaceutically acceptable salt, comprising contacting the bacterium with an therapeutically effective amount of the compound or its pharmaceutically acceptable salt.

21. The method of claim 20, wherein the contacting is performed in vitro or in vivo.

22. A method of treating a human or animal subject with the compound of claim 1 or its pharmaceutically acceptable salt, comprising administering or applying to the human or animal subject an therapeutically effective amount of the compound or its pharmaceutically acceptable salt.

23. A process for chlorinating an oxopyrido[2,3-d]pyrimidine with a phosphorus reagent, comprising:
providing an oxopyrido[2,3-d]pyrimidine and a phosphorus reagent, wherein the phosphorus reagent contains at least one chlorine atom;
suspending the oxopyrido[2,3-d]pyrimidine in the phosphorus reagent to form a reaction mixture;
heating and stirring the reaction mixture;
cooling the reaction mixture,
adjusting the reaction mixture to a basic pH; and
purifying the reaction mixture to obtain a chloropyrido[2,3-d]pyrimidine.

24. The process of claim 23, wherein the phosphorus reagent is selected from the group consisting of POCl₃, PCl₅, PCl₃, and CH₃Cl₂OP.

25. The process of claim 23, wherein the reaction mixture is heated to a temperature of from about 80 to about 150° C.

26. The process of claim 23, wherein the reaction mixture is heated for a time of from about 2 to 12 hours.

27. The process of claim 23, wherein the reaction mixture is heated under an inert gas.

28. The process of claim 27, wherein the gas is selected from the group consisting of argon and nitrogen.

29. The process of claim 23, wherein the reaction mixture is cooled by quenching the reaction mixture.

30. The process of claim 23, wherein the reaction mixture is adjusted to a basic pH of from about 8 to about 12.

31. The process of claim 30, wherein the reaction mixture is adjusted to a basic pH of about 10.

32. The process of claim 23, wherein the phosphorus reagent is POCl₃;
wherein the reaction mixture is heated to about 110° C. for about 2 hours; and
wherein the reaction mixture is adjusted to a basic pH of about 10.
